(19) United States
(12) Patent Application Publication
(10) Pub. No.: US 2020/0215726 A1
(43) Pub. Date: Jul. 9, 2020

(54) LIQUID-HEATED MOLD AND METHOD OF USING SME

(71) Applicant: KONINKLIJKE PHILIPS N.V., EINDHOVEN (NL)

(72) Inventors: RICHARD THOMAS HAIBACH, Verona, PA (US); Anthony Vincent Startare, Belle Vernon, PA (US); Gregory John Jablonski, Butler, PA (US)

(21) Appl. No.: 16/615,837

(22) PCT Filed: May 15, 2018
(86) PCT No.: PCT/EP2018/062442

§ 371 Date: Nov. 22, 2019

Related U.S. Application Data
(60) Provisional application No. 62/510,383, filed on May 24, 2017.

(51) Int. Cl. B29C 33/06; B33Y 80/00; B29C 35/04; B29C 35/08; B29C 33/04; A61M 16/06

(52) U.S. Cl. CPC B29C 33/06; B33Y 80/00; B29C 35/041; B29K 2083/00; B29C 33/04; A61M 16/06; B29C 2035/0855; B29C 35/0805

(57) ABSTRACT

The present system is configured to absorb, using a fluid medium in a reservoir, electromagnetic radiation to heat the fluid medium. The fluid medium may then conduct heat into a mold cavity formed by interior mold surfaces of at least two mold pieces. The fluid medium may heat up to, but not beyond, a phase transition temperature of the fluid medium, reducing instances of accidental damage to moldable material in a mold cavity during thermally-accelerated curing of the moldable material. In some instances a mold may be placed in liquid contained by the reservoir. In some instances, a mold may have integral reservoirs in individual mold pieces.

### CROSS-REFERENCE TO RELATED APPLICATIONS

This patent application claims the priority benefit under 35 U.S.C. § 119(e) of U.S. Provisional Application No. 62/510,383, filed on May 24, 2017, the contents of which are herein incorporated by reference.

### BACKGROUND

### 1. Field

The present disclosure pertains to a system and method for molding thermo-sensitive materials.

### 2. Background of the Invention

Molding thermo-sensitive materials to achieve a shape and characteristics of a final molded part may take long periods of time in implementations of curing that are performed without addition of external heat. Curing materials with the addition of external heat may result in uneven or excessive thermal exposure of the mold and molded material to the external heat, resulting in irregular molded parts that do not meet specification. Microwaves may be able to heat molds effectively, but without sufficient control on the time and power of microwave application, they can also heat the molds excessively.

### SUMMARY

The invention addressing these and other drawbacks relates to methods, apparatuses, and/or systems for prioritizing retrieval and/or processing of data over retrieval and/or processing of other data.

Some aspects of the present disclosure relate to a system for curing a moldable material. The system includes a mold comprising internal mold surfaces forming a mold cavity; a reservoir configured to hold a liquid in contact with surfaces of the mold outside the mold cavity such that an energy source configured to heat the reservoir heats the liquid placed in the reservoir.

Other aspects of the present disclosure relate to a method for curing moldable material. The method comprises adding moldable material to a mold cavity formed by internal mold surfaces of a mold; placing a liquid against surfaces of the mold outside the mold cavity; and heating the liquid with an energy source so as to cause heat to transfer to moldable material in the mold cavity.

Another aspect of the disclosure relates to a system configured to cure a moldable material, comprising means for molding the moldable material, the means for molding including a mold cavity into which the moldable material can be placed and surfaces outside the mold cavity; means for holding a liquid in contact with the surfaces outside the mold cavity; and means for generating heat within the reservoir so as to heat the liquid placed in the reservoir.

These and other features of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise. In addition, as used in the specification and the claims, the term “or” means “and/or” unless the context clearly dictates otherwise.

### BRIEF DESCRIPTION OF THE DRAWINGS

The invention is illustrated by way of example, and not by way of limitation, in the figures of the accompanying drawing and in which like reference numerals refer to similar elements;

FIG. 1 depicts a schematic diagram of an embodiment of a mold configured with a liquid reservoir;

FIG. 2 depicts a cross-sectional diagram of an embodiment of a mold configured with a liquid reservoir;

FIG. 3 depicts a cross-sectional diagram of an embodiment of a stackable mold configured with a liquid reservoir;

FIG. 4 depicts a cross-sectional diagram of an embodiment of a mold used in conjunction with a liquid reservoir;

FIG. 5 depicts a cross-sectional view of embodiments of a mold surface;

FIGS. 6A-6C illustrate rear, top, and bottom plan views of a CPAP mask that has been molded in accordance with the teachings of the present disclosure; and

FIG. 7. illustrates method steps in accordance with the teachings of the present disclosure.

### DETAILED DESCRIPTION OF THE INVENTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the development of moldable articles, such as custom made CPAP masks, the articles may be formed out of moldable material that can be set into a shape by curing the moldable material in a mold having a mold cavity. After curing the moldable material, the article may be flexible or a rigid according to the material used to form the prototype article.

Some types of curing processes, including addition curing, may be accelerated by heating the moldable material. Materials that undergo accelerated curing upon heating may be called thermally sensitive moldable materials. Curing of such thermally sensitive moldable materials may occur at room temperature in a process called room temperature vulcanization (RTV). At room temperature, curing may occur on a timeframe ranging from hours to days. By heating the moldable material, the curing time of some thermally sensitive moldable materials may be reduced from hours/days to as little as several minutes. Moldable materials that may benefit from heating during a curing process may include silicone, polysiloxanes, including substituted polysiloxanes, cyanates, epoxies, and acrylates, for example.

In some implementations of heat-accelerated curing, the moldable material is heated in a multi-piece metal mold. Metal molds may tend to be expensive because of material and labor costs associated with their manufacture. Metal molds may be heated by baking. However, baking metal molds may lead to overheating of the moldable material, resulting in damage to the molded part. Further, metal molds may be prone to uneven thermal exposure of the moldable material during a heating and curing process: thin regions of the metal mold may heat more quickly than thick regions of the mold, resulting in moldable material near the thin regions reaching elevated temperatures faster, and for longer, than moldable material near the thick regions of the metal mold.

An alternative to baking a metal mold with moldable material is achieved by use of electromagnetic radiation that is used in the curing of a moldable material in a non-metal mold. Electromagnetic stimulation may include directing a stream of electromagnetic radiation toward a mold/moldable material to heat the mold and/or the moldable material. Exposing materials to microwave radiation is one way of heating materials by electromagnetic stimulation.

Electromagnetic stimulation may be faster than baking. However, like baking, electromagnetic stimulation (e.g., application of microwave radiation) may also be subject to overheating of the moldable material. Accidental overheating may result from exposing the mold and/or moldable material to electromagnetic radiation for an incorrect amount of time, and from exposing the mold and/or moldable material to an incorrect intensity of electromagnetic radiation during a heating and curing process.

Overheating and uneven thermal exposure of moldable material during a heating and curing process, whether by baking, electromagnetic stimulation, or some other heating method, may cause damage to molded parts. By way of a non-limiting example, pieces of a molded part may be included in the mold cavity during the process of heating and curing the moldable material. In a process called “overmolding,” a material is at least partially exposed to a mold cavity such that the moldable material surrounds some or all of the material. Added materials may provide strength to a molded article to reduce tearing or punctures of a molded article. Added materials may provide a directional preference in deformation of a molded article, such as a medical mask. Added materials may provide fastening locations for straps fitted to a molded article. One embodiment of a molded article having an added material may be a medical mask formed of cured silicone and having at least one ring-shaped polyurethane inset to retain an elastic strap for holding the mask against a user's face during sleep, or during the dispensing of aerosolized medication to a patient.

During heating and curing of moldable material, added materials may be subject to melting, burning, deformation, and discoloration because the added material may have less thermal resistance to the heat applied to the mold than the moldable material. By way of a non-limiting example, a silicone medical mask (e.g., a CPAP mask) with a polyurethane ring inset to accept an elastic strap may be formed by heating and curing silicone material with the polyurethane rings inset in the mold prior to addition of the silicone precursor materials. Silicone may thermally degrade at temperatures that are significantly higher (tens of degrees) than solid polyurethane ring insets in the mold. Thus, while the silicone curing rate may be greatly enhanced by raising the temperature of the mold to very high temperatures without damaging the silicone, the polyurethane ring insets may be melted, deformed, or otherwise damaged by overheating during a heating and curing of the molded part.

In accordance with one aspect of the present disclosure, a liquid with a known boiling point is added to the mold as the source of heat. The liquid will not heat beyond the boiling point. The fluid medium can be used to restrict the temperature change of the moldable material to avoid damage to the molded part during curing. Specifically, the fluid medium has a phase transition associated with melting or boiling of the fluid medium. Thus, adding more energy to the fluid medium at the temperature of the phase transition will serve to accelerate the rate of the phase transition rather than to increase the temperature of the fluid medium. Therefore, by adjusting the temperature of a phase transition of the fluid medium (by adjusting the chemistry of the fluid medium itself), a maximum temperature of the mold and the moldable material may be obtained during a heating and curing process. A fluid medium may, in some embodiments, be heated without undergoing a phase transition during a heating and curing process, and still remain below a temperature threshold for properly molding the part.

Adjusting a temperature of a phase transition may include adding a first quantity of a first component (such as a first liquid or a solute) to a second quantity of a second component (such as a second liquid). Thus, combinations of liquids and/or combinations of solutes and solvents may be generated to have a selected boiling point less than a temperature threshold associated with the moldable material. The temperature threshold associated with the moldable material may be an upper molding temperature, the temperature above which damage occurs. Adjusting a temperature of a phase transition may include selecting a pure liquid having a phase transition within a desired range of temperatures. Adjusting a temperature of a phase transition may include blending one or more liquids to adjust a boiling point of the mixture of liquids. Adjusting a temperature of a phase transition may include adding a predetermined amount of solute to a liquid solvent to modify a boiling point transition (boiling point elevation) of the solution.

The fluid medium may include a pure liquid such as water, ethylene glycol, alcohols, organic solvents, and other compounds that are liquids at or near room temperature. In an embodiment, the fluid medium may include alcohols such as methanol, ethanol, isopropyl alcohol, propyl alcohol, ethylene glycol, and propylene glycol. The fluid medium may include organic compounds such as glycerin, toluene, benzene, toluene, hexane, cyclohexane, acetic acid, and solutions thereof. In an embodiment, the fluid medium may include oils such as olive oil, peanut oil, vegetable oil, and other saturated and unsaturated hydrocarbons. The fluid medium may also include mixtures of liquids described previously.

In an embodiment, the fluid medium may include a mixture of water and ethylene glycol. The boiling point of the water/ethylene glycol solution may be adjusted between about 100° C. (the boiling point of 100% water) and about 197° C. (the boiling point of 100% ethylene glycol). Thus, by selecting a ratio of water and ethylene glycol, a maximum temperature of the fluid medium may be adjusted to regulate the maximum temperature to which a mold and a moldable material can be heated, reducing the likelihood of damage occurring during a curing process.

The fluid medium may also include a solid material that undergoes a phase transition during heating of a mold configured with the solid material in the liquid reservoirs thereof. For example, in an embodiment, the fluid medium may be solid at room temperature, undergo a phase transition (melting) upon a first degree of heating, and a second phase transition upon a second degree of heating. In an embodiment, a fluid medium may be a liquid at room temperature and undergo a single phase transition upon heating.

Molds may include two mold halves or multiple mold pieces that form an interior mold cavity. The mold pieces have interior surfaces forming the mold cavity as well as exterior surfaces outside the mold cavity. The mold cavity is formed when the mold pieces are brought together into a closed configuration. The interior surface of one mold piece may face one or more interior surfaces of other mold pieces in order to form the mold cavity. Moldable material may be added to the mold cavity by pouring, pressing, injecting, and other methods of placing the moldable material in the mold cavity to form an article. Exterior surfaces of the mold, outside the mold cavity, are surfaces that are exposed to (in contact with) the fluid medium that is heated by electromagnetic radiation.

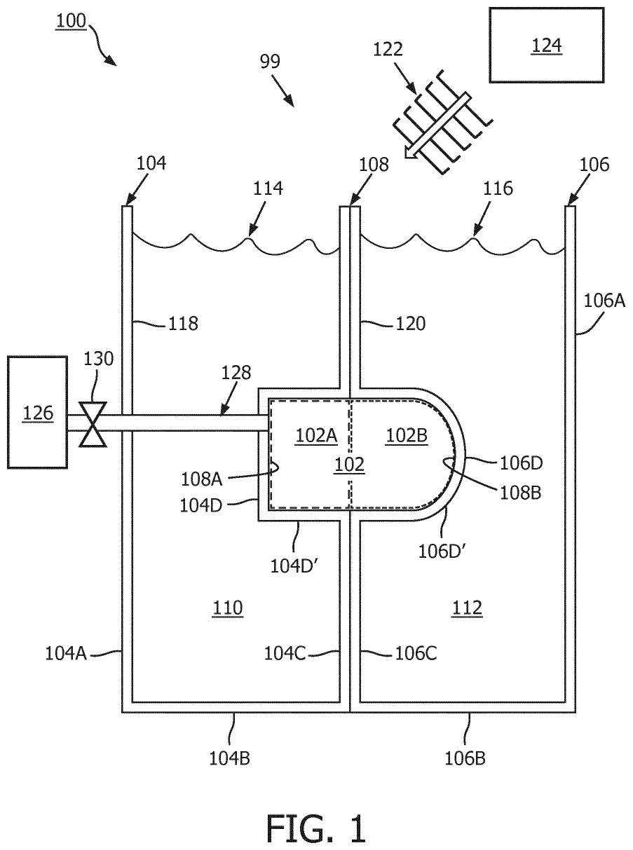

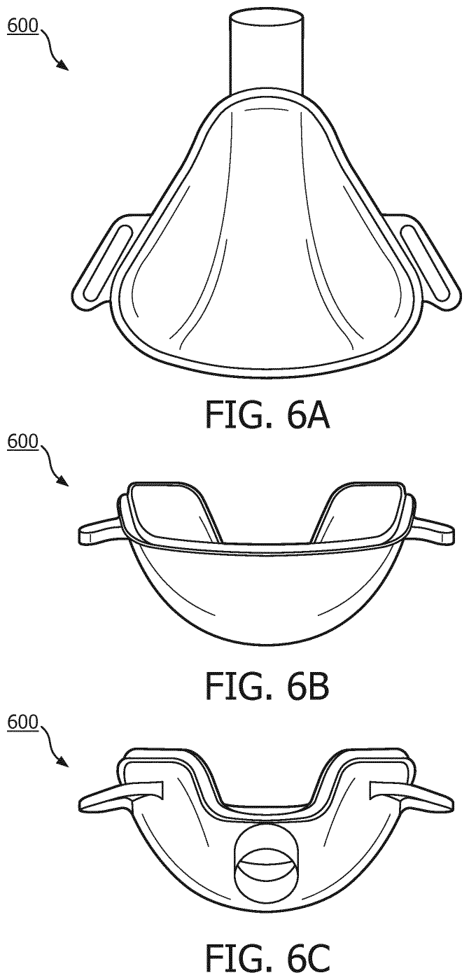

FIG. 1 depicts a vertical cross-sectional view of an embodiment of a mold system 100 in accordance with the present disclosure. The mold system 100 comprises a first mold piece 104 and a second mold piece 106. The mold system 100 is configured with at least one liquid reservoir (two reservoir's 110 and 112 used in this embodiment). FIG. 1 depicts that the reservoirs 110 and 112 each have side walls 104A (on first mold piece 104) and 106A (on second mold piece 106), respectively. The reservoirs 110 and 112 also have bottom walls, 104B and 106B, respectively. Each reservoir 110 and 112 has an open top into which liquid can be provided, as shown. The mold system 100 has a mold cavity 102 formed by a first cavity portion 102A in a first mold piece 104 and a second cavity portion 102B in a second mold piece 106. The mold cavity 102 is shown schematically. It should be appreciated that the mold cavity is configured to have the interior surfaces thereof conforming to the part to be molded. In one embodiment, the part to me molded is a CPAP mask 600, as illustrated in FIGS. 6A, 6B and 6C. The CPAP mask is formed from a silicone material. It should be appreciated, however, that many other different parts and/or products, made from other materials, can be molded in accordance with this disclosure.

The vertical cross-sectional view of FIG. 1 is through a middle portion of mold 100 showing mold cavity 102 and the walls of first mold piece 104 and the second mold piece 106. An interface 108 between first piece 104 and second piece 106 provides a seal between the two cavity portions 102A and 102B to ensure the moldable material placed into the mold cavity remains in the cavity is does not leak from the mold cavity 102. The interface 108 may comprise cooperating gasket and/or rubber surfaces 104E and 106E on the mold pieces 104 and 106, respectively. As shown, first piece 104 has an outer or side wall 104A, a bottom wall 104B, an interface wall 104C, and a mold cavity wall 104D. Second piece 106 has an outer wall 106A, a bottom wall 106B, an interface wall 106C, and a mold cavity wall 106D. Mold cavity 102 is formed by an inner mold surfaced 108A and 108B. Mold surface 108A is formed by the wall portion 104D of first mold piece 104 and a mold surface 108B is formed by the wall portion 106D of second piece 106. The wall portions 104D and 106D also have exterior surfaces 104D′ and 106D′, respectively. These exterior surfaces 104D′ and 106D′ of mold cavity 102 provide the surfaces against which liquid in the reservoirs 110 and 112 is held, such that heat imparted to the liquid can be conducted through the wall portions 104D and 106D to the part being molded in mold cavity.

As shown in FIG. 1, in one embodiment, a conduit 128 may be provided to supply moldable material to the mold cavity 102 from a mold material source 126. Optionally, one or more valves 130 (one shown for simplicity in FIG. 1) can be provided between the material source 126 and the mold cavity 102.

As shown, in one embodiment, the liquid reservoirs 110, 112 of the mold system 100 may be configured to completely surround the mold cavity 102 and the mold cavity walls 104D and 106D when filled with fluid medium. In an embodiment, first reservoir 110 may be filled with a first fluid medium 114 and second reservoir 112 may be filled with a second fluid medium 116. In an embodiment, first fluid medium 114 may the same as second fluid medium 116. In another embodiment, first fluid medium 114 may be different from second fluid medium 116. In one embodiment, one or more liquids with a known boiling point is added to the reservoirs as the source of heat. Because the one or more liquids will not heat beyond the boiling point, the temperature of the mold cavity will be maximized at that boiling point. In one embodiment, the surfaces of the reservoirs chamber are not smooth, so as to assure that the liquid medium does not superheat.

In various embodiments, the material forming the walls of the mold pieces 104 and 106 is made from a resin material, an organic compound or a plastic material. In any case, the material of the mold pieces will have a melting point higher than the boiling point of the liquid medium to be placed in the reservoirs 110, 112. Optionally, such materials of the mold pieces can be transparent to microwave radiation. In one embodiment, the walls are made from a material amenable to being formed by three dimensional printing.

In one embodiment, different fluid media placed within the reservoirs may have different phase transition temperatures, causing a one side of the mold to achieve a higher temperature than the second side of the mold. Differential heating of a mold cavity by using a different fluid medium in one or more liquid reservoir may be performed to adjust a cure rate of one portion of the mold cavity with respect to other portions of the mold cavity. A cure rate of one portion of the mold cavity may be adjusted based on a dimension of the mold cavity surrounded by the mold piece containing the different fluid medium in a liquid reservoir thereof. A cure rate in one portion of the mold cavity may be increased when a dimension of the mold cavity is large, with respect to other portions of the mold cavity, and a cure rate may be decreased when a dimension of the mold cavity is small, with respect to the other portions of the mold cavity.

The fluid medium within the reservoirs may be heated by electromagnetic stimulation using electromagnetic radiation 122 (e.g., microwave radiation) emitted by an energy source 124. Heating the fluid medium by electromagnetic radiation is then used to heat the material within the mold cavity 102, as heat from the fluid is conducted through the mold cavity walls 104D and 106D, which are in direct contact with both the fluid medium (on one side) and the moldable material (on the other side). The property of undergoing heating upon electromagnetic stimulation by electromagnetic radiation is known as “susceptance.” In one embodiment, the moldable material placed within the cavity 102 will have a relatively lower susceptance to microwave radiation, while the liquid medium(s) will have a relatively higher susceptance to microwave radiation, such the temperature/curing of the moldable material will not be largely impacted by the microwave radiation directly, but largely through the liquid medium. Moldable materials that do not heat upon exposure to electromagnetic radiation may thus be heated by the fluid medium in the liquid reservoir.

The energy source 124 that emits electromagnetic radiation 122 may comprise a microwave generator, such as those used in microwave ovens. In one embodiment, the energy source is provided separately from the mold pieces. For example, in one embodiment, the energy source 124 may form part of an enclosed microwave box (e.g., as a microwave oven) into which the mold pieces 104 and 106 may be placed. In other embodiments, other energy sources, using other wavelengths of electromagnetic radiation, may also be used to stimulate heating of the fluid medium in a liquid reservoir. In some embodiments, an energy source is a microwave generator. In one embodiment, the microwave generator emits microwave radiation having a frequency ranging from about 3000 MHz (megahertz) to about 500 MHz, although other radiation frequencies are envisioned. Some embodiments of an energy source generate microwave radiation with a wavelength between about 8 cm (centimeters) and about 45 cm. Some embodiments of energy sources used for heating a fluid medium may generate between 100 and 5000 W (watts) of electromagnetic radiation during operation of the energy source, although other delivered-power levels are envisioned. A mold with a reservoir may be placed within a chamber into which electromagnetic radiation is emitted for absorption by the liquid and moldable material.

As mentioned previously, the property of undergoing heating upon electromagnetic stimulation by electromagnetic radiation is known as susceptance. In some implementations of curing processes, mold material may have a susceptance lower than about 0.0001° C./(W*s*cm3). In some implementations of curing processes with liquid reservoirs, a ratio of susceptance of liquid divided by susceptance of mold material ranges from about 4.0 to about 0.5, although other values of the susceptance ratio may apply as new materials are developed for mold materials compatible with, e.g., three-dimensional printing. In some instances, susceptance of a mold material in a hot zone and/or a cold zone may range from about 0.0003° C./(W*s*cm3) to about 0.00001° C./(W*s*cm3). Susceptance of a mold material and a liquid may relate to the frequency of electromagnetic radiation to which the mold material and liquid are exposed during electromagnetic stimulation, thus, other ratios and values of susceptance are possible according to the response of a material to electromagnetic stimulation.

A susceptance of a mold material may be equal to or less than the susceptance of a liquid in a reservoir of mold pieces. In an embodiment where the susceptance of the mold material is greater than the susceptance of liquid a liquid reservoir, reservoir walls may be configured to experience thermal dissipation into the liquid to maintain the temperature of the mold piece and the temperature of the fluid approximately the same temperature during and after heating by electronic stimulation. The condition of having approximately the same temperature may apply to mold walls and fluid both during heating and at the phase transition temperature of the fluid. Thus, according to some embodiments, the susceptance of the liquid in a liquid reservoir may be between about 0.75 and about 2 times greater than the susceptance of the mold material. Electromagnetic susceptance of the mold material may be less than the susceptance of the liquid in the reservoir in order to avoid unintentional heating of moldable material caused by the mold, and not the liquid in the liquid reservoir surrounding the mold.

The liquid reservoirs 110, 112 may be filled with water, a mixture of water and ethylene glycol and/or other materials. Mold 100 is configured such that electromagnetic stimulation of the fluid medium-filled reservoir 110, 112 in a microwave oven (for example) may elevate the temperature of the mixture of water and ethylene glycol. The heated water/ethylene glycol mixture may then conduct heat through the mold material into the polysiloxane precursor materials to accelerate curing of the polysiloxane precursor material in the mold cavity. Heating of the fluid medium (mixture of water and ethylene glycol) may proceed up to the boiling point of the mixture of water and ethylene glycol. Excess energy added to the mold system 100 during a heating and curing process may serve to accelerate the phase transition of the fluid medium, but not to increase the temperature of the mold cavity 102 while fluid medium remains in the liquid reservoirs 110 and 112 surrounding the mold cavity 102.

In one embodiment, surfaces 118 of the liquid reservoirs 110 and 112 that contact the liquid medium are configured to promote a phase transition of the fluid medium during a heating and curing process. By way of a non-limiting example, first piece 104 may have a surface 118 within first reservoir 110 that is textured, roughened or provided with small bumps to promote boiling of a liquid during a heating and curing process. Similarly, second piece 106 may have a similar surface 120 within second reservoir 112 configured to promote boiling of a fluid medium during a heating and curing process. Phase transition promotion may be desirable during the heating and curing process because a liquid fluid medium may experience “superheating” or “bumping” during a phase transition. Superheating may involve localized elevation of the temperature of the fluid medium above the phase transition temperature as portions of the fluid medium do not undergo the phase transition (typically boiling) due to the effects of surface tension at the interface between the fluid medium and the surface of the mold in contact with the fluid medium.

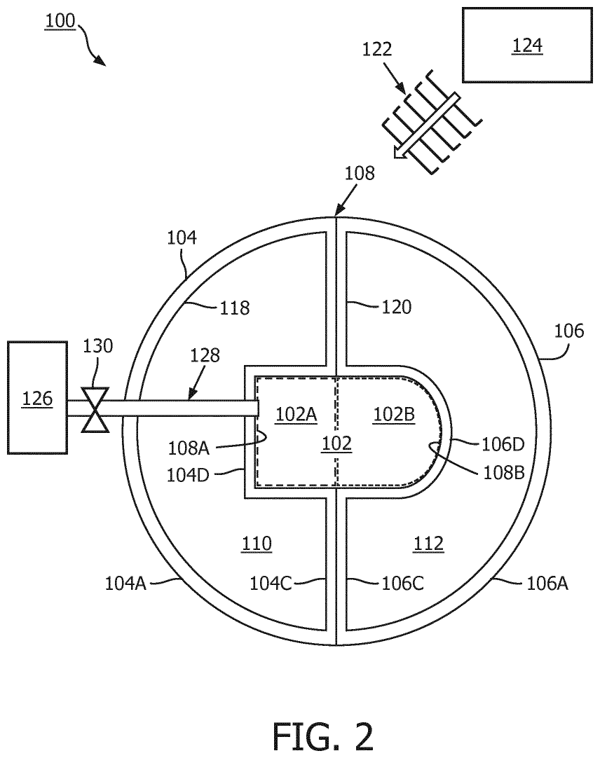

FIG. 2 depicts a horizontal cross-sectional view of mold 100 of FIG. 1. FIG. 2 shows cross-sectional view of mold 100 through mold cavity 102 and the mold pieces 104, 106. As noted previously, mold 100 has first liquid reservoir 110 and second liquid reservoir 112 bounded by cavity walls 104A and 106A. Interface 108 is shown on the sides of mold cavity 102 between the liquid reservoirs. Elements of FIG. 1 that are repeated in FIG. 2 are illustrated by the same reference numbers. Other embodiments of molds, including molds with different numbers of pieces, different shapes of liquid reservoirs, different heights, different numbers and shapes of mold cavities, are also envisioned by the present specification and are included, despite some limitations that apply to the specific description of mold 100 given herein.

As previously noted, embodiments of molds pieces constructed in accordance with the teachings of this disclosure may include mold pieces formed by three-dimensionally printing (3D printing) processes. A 3D printed mold may be formed directly from a digital file during an additive process where one or more mold materials are deposited together to form a mold piece. A 3D printing process may provide greater control over dimensions of a mold piece while forming interior surfaces that form the mold cavity than traditional metalworking processes such as computerized milling. 3D printing materials may be customized or selected or blended in a manufacturing process to adjust properties of a 3D printed mold piece to suit a particular curing process specification. Selectable properties of 3D printed mold pieces may include thermal conductivity of the mold pieces, specific heat of the mold pieces, dimensions of the mold in proximity to portions of the mold cavity to influence heat retention of the molded material in the mold cavity during, and after, a heating and curing process. Selectable properties of the mold pieces may be adjusted to improve the heating of the moldable material in the mold cavity without introducing uneven curing rates.

Thermal conductivity of moldable materials may be relevant to the manufacture of mold pieces used with liquid reservoirs because the heated liquid in a liquid reservoir adds heat to the moldable material by conduction through the mold piece. Thus, low thermal conductivity materials may be less suitable than high thermal conductivity materials in making molds used in conjunction with liquid reservoirs. In one embodiment, thermal conductivity of a mold piece with an integral liquid reservoir may be at least about 0.05 Watts/(m*K) in order to effectively heat moldable material in reasonable timeframes for curing materials. According to some embodiments, the thermal conductivity of a material of a mold piece may be about 0.2 W/(m*K) and range up to about 50 W/(m*K) with inclusion of thermally conductive filler materials in mold material. Interior and exterior surfaces of a mold piece formed by 3D printing processes may have tailored surface finishes to promote properties such as smooth release of molded material and phase transition promotion to reduce superheating of the fluid medium.

Some embodiments of heating and curing processes may heat moldable materials may be heated to temperatures of between about 50° C. and about 200° C. without harming the moldable material. In an embodiment, curing of RTV-2 silicone is accomplished by heating a fluid medium to a curing temperature between about 75° C. and about 100° C. for a curing time of up to three minutes. In an embodiment, liquid injection molded (LIM) silicone may be cured by heating a fluid medium to a curing temperature of about 150° C. for a curing time of up to three minutes. Curing temperatures and curing times of a mold system having moldable material therein may be related to the power output of electromagnetic stimulation of the mold system and the total mass of material (mold material and moldable material) being heated. Curing times of up to five minutes may be anticipated for many common embodiments of mold systems with commercially available microwave generator cavities.

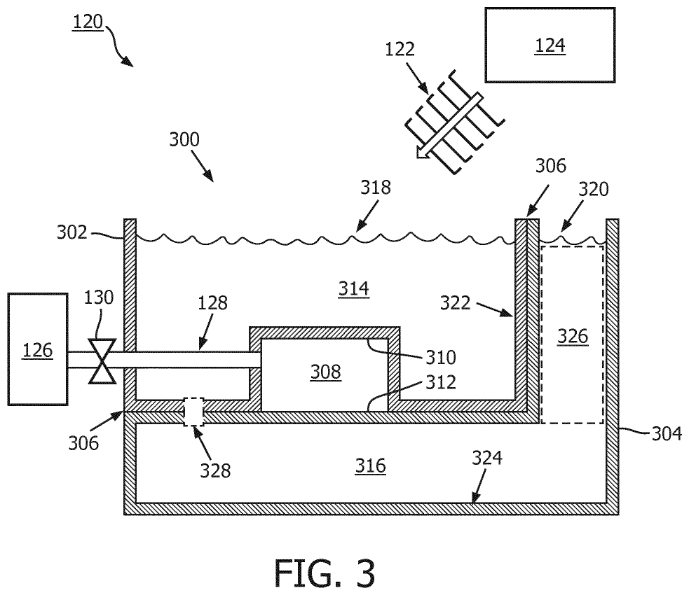

FIG. 3 depicts an embodiment of a stackable mold system 300. Mold system 300 has first piece 302 stacked on top of second piece 304. Interface 306 is between first piece 302 and second piece 304. Mold cavity 308 is formed between first interior surface 310 of first piece 302, and between second interior surface 312 of second piece 304. First piece 302 includes a first liquid reservoir 314 filled with a first liquid 318, and second piece 304 includes a second liquid reservoir 316 with a second liquid 320.

In one embodiment, an interior surface 322 of first reservoir 314 may be provided with a roughness, texture, or bumps to promote phase transition of the first liquid 318, and an interior surface 324 of second reservoir 316 is provided with a roughness, texture or bumps configured to promote phase transition of the second liquid 320. Liquid is provided to the second reservoir through narrowed neck region 326 serving as an opening for receiving the liquid medium. As in the first embodiment of FIGS. 1 and 2, the opening for receiving fluid for both reservoirs 314 and 316 are positioned above the upper surface of the mold cavity 308 (or 102 in FIG. 1). In that way, liquid will remain in contact with the upper surface of the mold cavity even if a substantial amount of the liquid is boiled off.

In some embodiments, first reservoir 314 and second reservoir 316 may be interconnected by one or more optional holes 328 between the reservoirs. Interconnection of reservoirs may serve to promote even heating of liquid in the reservoirs of mold 300 and provide more uniform heating of the mold cavity 308. In addition, in such embodiment, liquid need only be filled into one of the reservoirs and be communicated to the other reservoir through the holes 328. As in the prior embodiment, mold 300 may be heated by an energy source 124 (such as a microwave radiation generator) that emits electromagnetic radiation 122 (e.g., microwave radiation) to stimulate heating of liquid in the liquid reservoirs.

In the embodiment of FIGS. 1 and 2, the walls forming liquid reservoir 110, 112 were integrally formed with the walls forming the mold cavity 102. Otherwise stated, for example, in embodiments where the mold pieces 104 and 106 are formed by three dimensional printing, only two parts are printed. One part forming the reservoir 110 and mold cavity portion 102A, and a second part forming the reservoir 112 and mold cavity portion 102B. The integrally formed construction of FIGS. 1 and 2 also applies to the embodiment of FIG. 3, as can be appreciated by the FIG. 3, as can be appreciated by the figure.

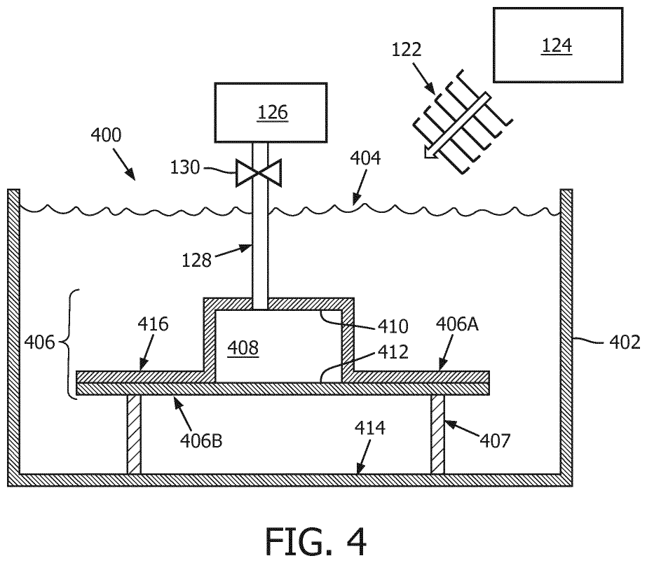

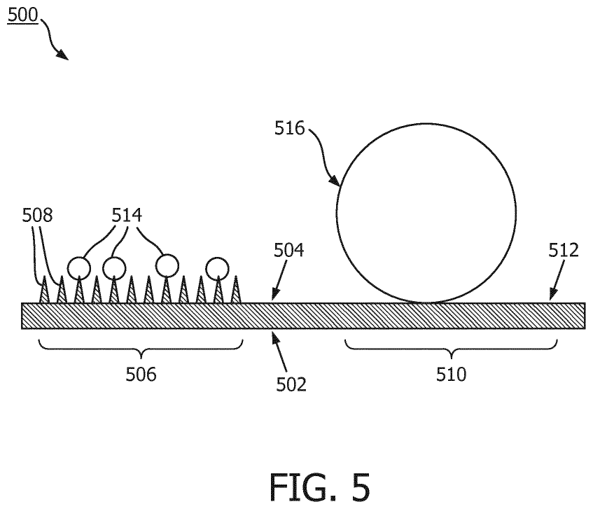

FIG. 4 depicts a cross-sectional diagram of an embodiment of a mold system 400. Mold system 400 does not have an integral liquid reservoir like the systems depicted in FIGS. 1-3. Rather, mold system 400 includes a mold assembly 406 that is constructed separately from, and can be placed within (and optionally removed from) the reservoir 402. In the embodiment shown, the mold assembly 406, and particularly mold cavity 408 thereof, is entirely immersed in a fluid medium 404 contained in the separately provided reservoir 402. Mold 406 includes a first piece 406A and a second piece 406B. First piece 406A has interior surface 410, and second piece 406B has interior surface 412 that cooperate to form mold cavity 408. Although not illustrated in FIG. 5, the system 400 has the same mold material source 126 (containing liquid or molten mold material to be molded into the part), conduit 128, and one or more valves 130 as shown in the prior embodiments. Mold 406 is separated from the interior walls of removable reservoir 402 by positioning posts 407. Positioning posts 407 allow fluid medium 404 to surround outer surfaces of mold 406 for even distribution of heat into mold cavity 408. The interior surface 414 is textured, roughened and/or provided with the bumps as previously described.

Mold system 400 may be placed within (as an oven) or adjacent to an energy source 124 that emits electromagnetic radiation 122 to heat the fluid medium as previously described. In an embodiment, the energy source 124 forms part of a microwave oven that emits microwave radiation. It is contemplated, as with the prior embodiments, that the energy source 124 can be considered the whole oven itself into which the reservoir 402 can be placed, or alternatively can be considered for the purpose of this disclosure, a microwave generator that can be placed adjacent the reservoir 402.

In some embodiments, the fluid medium 404 may be preheated before the mold assembly 406 is placed in the reservoir 402. Optionally, the reservoir 402 may be configured (large enough) to receive a plurality of mold assemblies in the fluid medium 404 to accommodate curing of a plurality of molded parts.

As mentioned previously, in one embodiment, surfaces of the liquid reservoirs disclosed herein (such as surfaces 118,120, 322, 324 and 414 referenced above) may have a roughened or textured surface to prevent superheating of the fluid therein. FIG. 5 depicts a segment of an embodiment of a mold 500 having such textured roughened or bumpy surfaces that are configured to promote a phase transition, as may be used in the liquid reservoirs of any of the above described embodiments. For example, mold 500 has a mold wall 502 with an interior surface 504 where a first region 506 of interior surface 504 has a plurality of nucleation sites 508, and a second region 510 of interior surface 504 has a smooth surface 512. Smooth surface 512 may be a new surface that is free of nucleation sites, or a surface that has fewer nucleation sites than are found in first region 506. A nucleation site such as nucleation site 508 may comprise bumps, points, and/or a roughened or textured configuration (nucleation sites), or the like. The interior surface 504 extends into the reservoir so that it contacts the liquid. The nucleation sites 508 are configured to promote bubble formation during heating of the liquid. The nucleation sites are configured to lower the surface tension of the liquid, promoting formation of small bubbles 514.

In the absence of nucleation sites on a surface, such as in second region 510, surface tension of the liquid would be higher than in the fluid against nucleation sites 508. Thus, the degree of superheating may be greater than in the liquid above first region 506 and when a bubble 516 forms above smooth surface 512 in second region 510, the bubble 516 formed may be larger than small bubbles 514. Because of larger surface tension and larger (potential) degree of superheating, bubble 516 may also form more rapidly in the liquid than small bubble 514, reducing the chance of liquid displacing around the large bubble 516 as it exits the liquid. Thus, in one embodiment, smooth surfaces 512 would not be positioned to be in contact with the liquid within reservoir. However, that is not to say that the present disclosure cannot use one or more smooth surfaces within the reservoir, as such embodiments are contemplated as well, as other mechanisms for preventing superheating of fluids are known in the art.

In some embodiments, a surface of a liquid reservoir is provided with the nucleation sites 508 (integral nucleation sites) during formation of the reservoir 402, such as during three-dimensional (3D) printing processes used to manufacture the reservoir 402. In some embodiments, nucleation sites 508 may be formed on a mold surface by abrading the mold surface. In some embodiments, rather than using mold pieces with integral nucleation sites, extrinsic nucleation sites may be added to a liquid by adding a phase transition promotor to a liquid reservoir. A phase transition promotor may include objects with rough surfaces, and may further include boiling chips (pieces of calcium carbonate), ceramics, scratched glass, or chemically-etched glass.

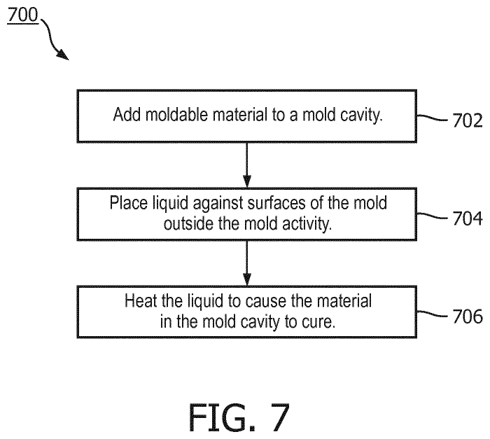

FIG. 7 illustrates a method for curing moldable material. The system comprises a part forming cavity, internal surfaces of a mold, and/or other components. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 7 and described below is not intended to be limiting.

At an operation 702, moldable material is added to a part-forming cavity formed by internal mold surfaces of a mold. In some embodiments, operation 702 is performed by surfaces the same as or similar to surfaces 108A and B (shown in FIG. 1 and described herein).

At an operation 704, a liquid is placed against surfaces of the mold outside the mold cavity. In some embodiments, operation 704 includes adding a phase transition promoter to the liquid. In some embodiments, operation 704 includes incorporating a phase transition promoting texture in a surface of the mold outside the mold cavity. In some embodiments, operation 704 includes adding the liquid to an integral liquid reservoir of the mold. In some embodiments, operation 704 is performed by reservoirs 110 and 112 (shown in FIG. 1 and described herein).

At an operation 706, the liquid is heated with an energy source so as to cause heat to convectively transfer to moldable material in the part-forming cavity. In some embodiments, operation 706 includes determining a temperature window of the mold and moldable material by selecting a liquid having a boiling point less than or equal to a threshold temperature. In some embodiments, operation 706 includes heating the liquid with an energy source comprises generating electromagnetic radiation configured to be absorbed by the liquid. In some embodiments, operation 706 is performed by energy source 124 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A mold system configured to cure a moldable material, comprising:
a mold comprising internal mold surfaces forming a mold cavity; and
a reservoir configured to hold a liquid in contact with surfaces of the mold outside the mold cavity such that an energy source configured to heat the reservoir heats the liquid placed in the reservoir, wherein the mold is formed by 3D printing processes.

2. The system of claim 1, wherein a material forming the mold has a susceptance to microwave radiation that is less than a susceptance of the liquid to be placed in the reservoir.

3. The system of claim 1, wherein the energy source is a microwave generator.

4. The system of claim 2, wherein the mold is made of a material having a thermal conductivity greater than 0.05 Watt/(meter*Kelvin).

5. The system of claim 1, wherein the mold system comprises two or more mold pieces that cooperate to form the mold cavity, and wherein at least a portion of the mold cavity is formed integrally with the reservoir.

6. The system of claim 1, wherein the reservoir configured to hold a liquid in contact with surfaces of the mold outside the mold cavity is further configured to retain an entirety of the mold within the liquid.

7. The system of claim 1, wherein the reservoir in which the liquid is held has nucleation sites formed on the surface in contact with the liquid to reduce a surface tension of the liquid to thereby promote a phase transition of the liquid.

8. The system of claim 1, further comprising nucleation sites within the reservoir.

9. A method for curing moldable material, comprising:
providing a mold comprising internal mold surfaces forming a mold cavity;
adding moldable material to the mold cavity;
placing a liquid against surfaces of the mold outside the mold cavity; and
heating the liquid with an energy source so as to cause heat from the liquid to convectively transfer to the moldable material in the mold cavity,
wherein the mold is formed by 3D printing processes.

10. The method of claim 9, further comprising adding a phase transition promoter to the liquid.

11. The method of claim 9, wherein the heating comprises applying microwave radiation to the liquid.

12. The method of claim 9, further comprising determining a composition of the liquid to be placed against the surfaces of the mold based upon at least one characteristic of the moldable material.

13. The method of claim 12, wherein the at least one characteristic comprises an upper molding temperature threshold of the moldable material.

14. A system configured to cure a moldable material, comprising:
means for molding the moldable material, the means for molding including a mold cavity into which the moldable material can be placed and surfaces outside the mold cavity;
means for holding a liquid in contact with the surfaces outside the mold cavity; and
means for generating heat within the means for holding so as to heat the liquid placed in the means for holding,
wherein the means for molding the moldable material are formed by 3D printing processes.

15. The system of claim 14, wherein the means for generating heat comprises a microwave energy source.

16. The system of claim 14, wherein the means for holding comprises means for promoting a phase transition of the liquid.
